(19) United States
(12) Patent Application Publication
(10) Pub. No.: US 2022/0246232 A1
(43) Pub. Date: Aug. 4, 2022

(54) METHOD FOR DIAGNOSING DISEASE RISK BASED ON COMPLEX BIOMARKER NETWORK

(71) Applicant: KOREA ADVANCED INSTITUTE OF SCIENCE AND TECHNOLOGY, Daejeon (KR)

(72) Inventors: DongHo CHO, Daejeon (KR); Dong Jin JI, Daejeon (KR)

(21) Appl. No.: 17/168,288

(22) Filed: Feb. 5, 2021

(30) Foreign Application Priority Data
Feb. 3, 2021 (KR) 10-2021-0015125

(51) Int. Cl. G16B 5/20; G16B 40/00

(52) U.S. Cl. CPC G16B 5/20; G16B 40/00

(57) ABSTRACT

Provided is a method for diagnosing a disease risk based on a complex biomarker network. More particularly, provided is a method for predicting or diagnosing a disease risk by constructing a complex disease relation network from biomarkers extracted from a liquid biological specimen and automatically extracting a disease marker from the complex disease relation network. It was confirmed that the method for predicting or diagnosing a disease risk using the biomarkers extracted from the liquid biological specimen developed according to the present invention applies an improved network analysis and learning method as compared to conventional methods, and thus enables disease-related diagnosis which shows high sensitivity and specificity even when only a few biomarkers are applied, which indicates that the method of the present invention shows superior extraction performance, compared to the methods using conventional variational dropout-based biomarker extraction.

### CROSS-REFERENCE TO RELATED APPLICATIONS

This application claims priority under 35 U.S.C. § 119 to Korean Patent Application No. 10-2021-0015125, filed on Feb. 3, 2021, in the Korean Intellectual Property Office, the disclosure of which is incorporated herein by reference in its entirety.

### TECHNICAL FIELD

The following disclosure relates to a method for diagnosing a disease risk based on a complex biomarker network, and more particularly, to a method for predicting or diagnosing a disease risk by constructing a complex disease relation network from biomarkers extracted from a liquid biological specimen and automatically extracting a disease marker from the complex disease relation network.

### BACKGROUND

In recent years, the technology for diagnosing a disease has been mostly researched for a method of measuring an expression level of a gene or a protein mainly using a microarray, and ardent research on single nucleotide polymorphisms (SNPs), copy number variations, and variations in base sequences such as insertion, deletion, or substitution of bases, and the like have also been conducted.

However, because the conventional technology as described above has been developed for the purpose of determining the correlation using one type of specimen and the disease, there is little or no method for analyzing a relation between various types of genetic variation information and the disease. Therefore, there is an urgent need for finding a solution to these problems because it is not easy to find novel disease-specific information, and problems regarding the low accuracy of a diagnostic method have come into the spotlight.

### RELATED ART DOCUMENT

### Patent Document

Patent Document 0001: Korean Patent Laid-Open Publication No. 2019-0137012 (Dec. 10, 2019)

### SUMMARY

An embodiment of the present invention is directed to providing a method capable of extracting disease-related biomarkers from a liquid biological specimen and predicting a risk of the corresponding disease from disease markers derived from a network for the disease-related biomarkers.

In a general aspect, a method for predicting a disease risk through analysis of the relation between the types of genetic information includes extracting types of complex genetic information from specimens of a patient and a normal person; constructing a complex genetic information library through information comparison/analysis between the types of complex genetic information; extracting disease-specific biomarkers from the complex genetic information library; and constructing a network model for predicting a disease risk from the disease-specific biomarkers and predicting a disease risk.

In the method for predicting a disease risk through analysis of the relation between the types of genetic information, the types of complex genetic information may include information on expression of genetic information of any one or two or more selected from the group consisting of DNAs, RNAs, and proteins, or information on syntheses thereof.

In the method for predicting a disease risk through analysis of the relation between the types of genetic information, the complex genetic information library may be constructed by deducing the types of complex genetic information using a statistical analysis or optimization method.

When the disease-specific biomarkers are extracted, the method for predicting a disease risk through analysis of the relation between the types of genetic information may further secure information on a base sequence of the corresponding genetic information to extract genetic information variations including single nucleotide polymorphisms (including addition, deletion, or substitution of the base sequence), or copy number variations.

In the method for predicting a disease risk through analysis of the relation between the types of genetic information, the relation between a disease and the types of complex genetic information present in the complex genetic information library may be analyzed using an optimization method or learning method to extract a biomarker associated with the disease.

In the method for predicting a disease risk through analysis of the relation between the types of genetic information, a statistic disease network model or a dynamic disease network model may be constructed based on the disease-specific biomarkers.

In the method for predicting a disease risk through analysis of the relation between the types of genetic information, the optimization method may be selected from the group consisting of a simulated annealing method, a genetic algorithm, a tap search method, a simulated evolution, and a probabilistic evolution method.

In the method for predicting a disease risk through analysis of the relation between the types of genetic information, the learning method may be selected from the group consisting of a neural network and deep learning.

In the method for predicting a disease risk through analysis of the relation between the types of genetic information, the neural network may be selected from the group consisting of a convolutional neural network (CNN) and a recurrent neural network (RNN).

In the method for predicting a disease risk through analysis of the relation between the types of genetic information, an attention model using an attention layer may be applied upon extraction of the biomarkers to model a correlation between a full-length sequence and some certain sequences in the form of a matrix.

In the method for predicting a disease risk through analysis of the relation between the types of genetic information, the accuracy of the predicted disease risk may be such that sensitivity and specificity to the 20 to 35 extracted biomarkers is greater than or equal to 80%, respectively.

In the method for predicting a disease risk through analysis of the relation between the types of genetic information, the disease may be caused by cognitive function- and/or memory-related impairments.

In the method for predicting a disease risk through analysis of the relation between the types of genetic information, the disease may be Alzheimer's disease, Huntington's disease, Parkinson's disease, or amyotrophic lateral sclerosis.

A disease-specific biomarker extracted by the method for predicting a disease risk is provided.

### BRIEF DESCRIPTION OF THE DRAWINGS

The above and other objects, features and advantages of the present invention will become more apparent to those of ordinary skill in the art by describing in detail exemplary embodiments thereof with reference to the attached drawings, in which:

FIG. 1 shows a flowchart for stepwise learning and extraction in a useful biomarker automatic extraction algorithm based on machine learning;

FIG. 2 shows a flowchart for attention matrix and output computation in an attention layer;

FIG. 3 shows a flowchart regarding extraction of biomarkers; and

FIG. 4 shows an algorithm for predicting a disease risk using a disease risk prediction network according to the present invention.

### DETAILED DESCRIPTION OF EMBODIMENTS

Hereinafter, a method for diagnosing a disease risk based on a complex biomarker network according to the present invention will be described in detail with reference to the accompanying tables and drawings.

When the drawings are presented hereinbelow, the drawings are shown as one example to sufficiently provide the scope of the present invention to those skilled in the art. Therefore, it should be understood that the present invention may be embodied in various forms, but is not intended to be limiting in the drawing presented hereinbelow. In this case, the drawing presented hereinbelow may be shown in an exaggerated manner to make the scope of the present invention more clearly apparent.

Although the terms first, second, etc. may be used to describe various elements, these elements are not limited by these terms. These terms are only used to distinguish one element from another. For example, a first element could be termed a second element, and, similarly, a second element could be termed a first element, without departing from the scope of the present invention.

In this case, unless otherwise defined, the technical and scientific terms used herein have the same meaning as commonly understood by one of ordinary skill in the art to which the present invention pertains. In the following description and the accompanying drawings, a description of known functions and configurations, which may unnecessarily obscure the subject matter of the present invention, will be omitted. The terms as defined in the dictionaries generally used in the art should be construed as having the same meaning as that in the context of the related art, and should not be construed as having an ideal meaning or overformal meaning, unless clearly defined in the present application.

Also, the singular forms “a,” “an,” and “the” used in the specification of the present invention and the appended claims may be intended to refer to those including plural referents unless the context clearly dictates otherwise.

In addition, the units used without any particular comments in the specification of the present invention are based on weight. For example, the units of % or percentage refer to a percent (%) by weight or weight percentage.

Further, in the specification of the present invention, the expression “comprise(s)” is intended to be an open-ended transitional phrase having an equivalent meaning to the expressions “contain(s),” “include(s),” “have,” “has,” and “is(are) characterized by,” and does not exclude elements, materials, or steps, all of which are not further recited herein. Also, the expression “consist(s) essentially of” means that one element, material or step, which is not recited in combination with the other elements, materials, or steps, may be present at an amount having an acceptably significant influence on at least one basic and novel technical idea of the present invention. Also, the expression “consist(s) of” means the presence of only the elements, materials or steps defined hereafter.

The terms “component,” “composition,” “compound composition,” “compound,” “drug,” “pharmaceutically active agent,” “active agent,” “cure,” “therapy,” “treatment,” and “agent” used in the specification of the present invention are used interchangeably to refer to a compound or compound(s), or a material composition that induces a desired pharmaceutical and/or physiological effect by means of a local and/or systemic action when administered to a subject (a human or an animal).

In the present invention, the term “sample” or “specimen” refers to a subject to be analyzed, and thus is used as having the same meaning throughout the specification.

A method for predicting a disease risk through analysis of the relation between the types of genetic information according to the present invention includes extracting types of complex genetic information from specimens of a patient and a normal person; constructing a complex genetic information library through information comparison/analysis between the types of complex genetic information; extracting disease-specific biomarkers from the complex genetic information library; and constructing a network model for predicting a disease risk from the disease-specific biomarkers and predicting a disease risk.

According to the present invention, information on general life phenomena and diseases may be compared and analyzed, and then distinguished based on the extracted complex genetic information to aid in understanding the genetic information functions, and further construct a model for deducing disease-specific biomarkers having high accuracy and predicting a disease risk.

In the present invention, a big data processing method, an artificial intelligence-based deep learning method (e.g., a machine learning method), and the like may be combined and used for a great deal of genetic information in order to construct the model for deducing disease-specific biomarkers and predicting a disease risk.

First, a step of extracting types of complex genetic information from specimens of a patient and a normal person will be described in detail.

In the step of extracting types of complex genetic information from specimens of a patient and a normal person, information regarding DNAs, RNAs, proteins, and the like for the full-length genomes of the specimens may be secured. A method of acquiring the information is not limited as long as it does not hinder achievement of the objects of the present invention, but may, for example, be secured from the genetic information database provided throughout the world. As a more specific example, the database provided by the National Institutes of Health (NIH), and the like may be used. As an even more specific example, the data for European patients with Alzheimer's disease and mild cognitive impairments may be secured from the AddNeuroMed database (The article recited in Ann NY Acad Sci. 2009 October; 1180: 36-46) provided by the National Library of Medicine (U.S.A.) as information regarding the improvement of cognitive functions. As another example, genomes of specimen samples taken from a hospital or taken directly from patients are sequenced, thereby acquiring the information. As still another example, a whole exome sequence set that plays a direction role in the synthesis of proteins in a gene may be secured and used, but the present invention is not limited thereto.

In the present invention, genome sequence information of the specimen may have some changes according to the type of the genetic information database, the equipment used for sequencing, a sequencing method, and the like. Also, the genome sequence information is not limited as long as it does not hinder achievement of the objects of the present invention, but may, for example, be based on the information provided on a human genome map found in the human genome project.

In the present invention, the full-length genome sequence information of the specimens of the patient and the normal person may be information that becomes a basis for detection of the biomarkers according to the present invention, and may include DNA information such as cf-DNA, ct-DNA, and the like, RNA expression information such as mRNA, mi-RNA, and the like, information on the protein synthesis, and the like, all of which may be obtained from this genome sequence information, and thus performs analysis based on the difference present in the genome sequence information of the specimens. In the full-length genome sequence information, information on chromosomes, information regarding the positions of base sequences in the chromosomes, information on base sequence variations associated with the addition, deletion, or substitution of the base sequence, information on RNAs, information on protein expression, information including three-dimensional structures and reliability of proteins, and the like may be mainly used to detect biomarkers for diagnosing a disease, but the present invention is not limited thereto.

In the present invention, analysis of the information included in the genome sequence information may be performed by adding and subtracting the information according to the type, version, and service environment of a program used.

Next, a step of constructing a complex genetic information library through information comparison/analysis between the types of complex genetic information will be described in detail.

In the step of constructing a complex genetic information library through information comparison/analysis between the types of complex genetic information, a complicated relation present between the types of complex genetic information, which is obtained in the step of extracting types of complex genetic information from specimens of a patient and a normal person, may be analyzed to extract important genetic information associated with the disease, thereby constructing a library.

In the present invention, the genetic information is not limited as long as it does not hinder achievement of the objects of the present invention, but may, for example, include DNA information such as cf-DNA, ct-DNA, and the like, which are associated with a gene expression process, information on expression of RNAs such as mRNA, mi-RNA, and the like, information on synthesis of proteins.

To extract important genetic information factors to be analyzed, the method is not limited as long as it does not hinder achievement of the objects of the present invention, but may include a process as follow.

First, the single genetic information factors may be used to extract classification accuracy for what differentiate a normal group from a disease group. The type and number of the single genetic information factors are not limited when the normal group is differentiated from the disease group using the information only, but may, for example, include single nucleotide polymorphisms (including variations of a base sequence associated with the addition, deletion, or substitution of the base sequence), copy number variations, protein sequencing polymorphisms, and the like, but the present invention is not limited thereto. As one example, when variations of the base sequence appear commonly in a specimen sample of the disease group and the same variations do not appear in a specimen sample of the normal group, it is desirable to identify the corresponding genetic information, extract location information and variation information of the base sequence for the genetic information, and store the extracted information.

Then, an on/off tag which may measure a difference between an actual expression level and a reference level for each of the genetic information factors to determine whether the corresponding genetic information factors have an influence on selection of the disease group may be set. As one example of a method for setting the on/off tag as described above, reference values of the expression levels for the respective steps associated with an important hereditary gene expression process may be set to Th₁, Th₂, and Th₃ respectively, and may be used under the definition of increased reference values (Th₁^{up}, Th₂^{up}, and Th₃^{up}) and decreased reference values (Th₁^{down}, Th₂^{down}, and Th₃^{down}) respectively, when the expression levels of the genetic information are increased or decreased by the disease. By using the variables as defined above, it is possible to extract the genetic information whose expression level is changed by the disease while satisfying the requirements for each of the expression levels for the secured specimen samples. In this case, the information on the base sequence of the corresponding genetic information may be secured, and used when necessary. In this case, the aforementioned variations such as single nucleotide polymorphisms (including variation associated with the addition, deletion, or substitution of the base sequence), copy number variations, or the like may be extracted to make use of variation information of the base sequence by the disease, but the present invention is not limited thereto.

The correlation between the types of complex genetic information may be identified by using the extracted genetic information factors to analyze a change in expression level between the types of genetic information, a variation in base sequence, and the like, which correspond to different steps, to construct a library, and may be then used to deduce the biomarkers.

The information analyzed by the method may be converted into a predetermined platform, that is, a form of the same frame, which may be then stored or managed.

Subsequently, a step of deducing disease-specific biomarkers by subjecting the complex genetic information library to an optimization method or a learning method will be described in detail.

In the step of deducing disease-specific biomarkers by subjecting the complex genetic information library to an optimization method or a learning method, the complex genetic information library constructed by the method may be analyzed by an optimization method or a learning method to deduce biomarkers specific to the disease.

A method for extracting disease-specific biomarker candidates is not limited as long as it does not hinder achievement of the objects of the present invention, but may determine whether the relation between genetic information of the specimen sample in the disease to be proved and the same genetic information in the library is established from the deduced complex genetic information library and extract a relation between increase and decrease in the genetic information, variation information of the base sequence, and the number of genetic information to set them as a candidate group for deducing disease-specific biomarkers. For selection of the candidate group, it is preferably desirable for the disease-specific biomarkers to satisfy increasing the accuracy, which represents the corresponding disease, to the maximum extent and minimize the number of the genetic information to be considered as well. For this purpose, this may be defined in the form of multi-variable function optimization, and subjected to a mathematical algorithm to deduce disease-specific markers, but the present invention is not limited thereto.

The mathematical algorithm for multi-variable function optimization may be introduced and used without limitation as long as it is a method capable of solving the problems regarding the multi-variable functions. For example, the mathematical algorithm for multi-variable function optimization may include a simulated annealing method, a genetic algorithm, a tap search method, a simulated evolution, a probabilistic evolution method, and the like. Desirably, a genetic algorithm may be used. When the disease-specific biomarkers are extracted by the method, a whole process does not need to be necessarily finished. In this case, the process is stopped during calculation of an optimal solution, and the best solution among the solutions calculated up to that time may also be used.

The genetic algorithm has a basic theory on the biological genetics in the natural world, and is a method for gradually making better solutions by expressing possible solutions to the problems in a given form of data structure using a parallel global search algorithm, and then gradually transforming these solutions. Here, the data structure representing the solutions may be expressed as a gene, and a process for gradually making better solutions by transforming the solutions may be expressed as an evolution. In other words, the genetic algorithm may be referred to as a simulated evolution search algorithm to find a solution x which optimizes any unknown function Y=f(x). The genetic algorithm is closer to an approach for solving a certain problem rather than an algorithm for solving the problem, and thus may be applied to all the problems that may be paraphrased in the form that may be used in the genetic algorithm. In general, when a problem is excessively complicated to a non-computable extent, it is desirable because it may be approached as a plan for obtaining a solution closer to the optimal solution even when the optimal solution is not actually obtained through the genetic algorithm.

In the method for deducing disease-specific biomarkers, a learning sample to be analyzed, and a verification sample for verifying the accuracy of the learning sample may be provided. As one example, the verification sample may include only the corresponding disease-specific genetic information through the existing analysis, but the present invention is not limited thereto. As performed in one embodiment of the present invention, libraries to be analyzed may be optionally divided into a learning sample and a verification sample to perform learning. In this case, the learning process may be repeated several times to improve accuracy.

When the libraries are large in size, it is difficult to calculate classification accuracy for all subsets, and the libraries have high complexity. Therefore, it is desirable to perform a process of reducing the complexity. When the size of libraries is N, 2\hat{ }N numbers of cases are enrolled for the number of all the subsets. Therefore, as the size of the library increases, it is difficult to calculate the classification accuracy for all the subsets, and the library has high complexity. Therefore, to solve this problem, it is, for example, necessary to reduce the complexity using a heuristic algorithm, and the like. For example, when the size of subsets is N, the number of causes for the markers to be examined is reduced to N(N+1)/2 by confirming the probability of markers and gradually reducing the size of the subsets only under the preferential consideration of the markers having the highest probability.

Selection of the genetic information that is a variable for multi-variable function optimization is not limited as long as it does not hinder achievement of the objects of the present invention, but the genetic information may, for example, be optionally selected according to the heuristic algorithm. Preferably, a combination of genetic information having the highest accuracy may be selected. As one example, when genetic information mi-RNA1 and ct-DNA5 have a characteristic of increasing at the same time, information associates with an increase/decrease in expression level of each of the mi-RNA1 and ct-DNA5 may be used for learning so that they can be used as two features, respectively. In this case, whether the characteristic of the mi-RNA1 and ct-DNA5 of increasing at the same time is present in a sample may be used as one feature for learning.

In the present invention, the artificial intelligence-based deep learning method used for the learning in order to deduce the biomarkers is not limited as long as it does not hinder achievement of the objects of the present invention, but may, for example, be used for a neural network, deep learning, and the like. One example corresponding to the neural network may include a convolutional neural network (CNN), a recurrent neural network (RNN), and the like. For example, CNN may be used in one embodiment of the present invention, but the present invention is not limited thereto. A proper learning method may be selected and used according to the ensured data and the features of the biomarkers.

Then, a method for automatically extracting useful biomarkers based on machine learning will be described in detail in the present invention.

Because a test for predicting a disease risk based on the existing medicine method takes a long time to perform, and has an inconvenience of requiring invasive testing, it was intended to acquire information on biomarkers based on a liquid biopsy capable of performing a test using only a small amount of peripheral blood. For example, the information on biomarkers that may be extracted based on the liquid biopsy may include concentrations of proteins, a concentration of miRNA, the shape and number of blood cells, a cf-DNA sequence, and the like.

The information on biomarkers extracted based on the liquid biopsy possesses information on the health condition of the whole body, but has a drawback in that it is difficult to extract useful information because the information on biomarkers has an enormous amount of information. Therefore, a method for automatically extracting useful biomarkers using only an algorithm without any artificial human intervention was intended to be applied according to the present invention.

The information on biomarkers is generally in the form of a long sequence, and has a characteristic of having an enormous amount of information. As one example, it is common that DNA sequence information generally has a length of several tens of thousands of base pairs (bps). Among them, only a tiny fraction of the base pairs is generally associated with a disease. Also, there are several base pairs which are spaced apart from each other in a fraction of the base pairs associated with the disease. Therefore, it is important to make use of an algorithm capable of employing the characteristics of sparse information using a higher-order level of data to automatically extract the biomarkers.

In the existing field of machine learning, a representative example which deals with a long sequence includes natural language processing (NLP). The natural language processing is based on a process of recognizing and understanding a meaning underlying in sentences having various lengths, and thus has many similarities to processing of the information on biomarkers. In such natural language processing, a correlation between a full-length sequence and some certain sequences may be modeled so that it can be used as an attention model for interpreting a meaning of the full-length sequence.

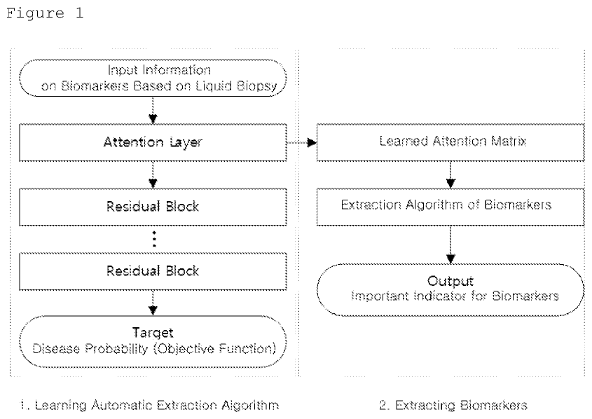

In the method for predicting a disease risk through analysis of the relation between the types of genetic information according to one embodiment of the present invention, an attention model using an attention layer may be applied upon extraction of the biomarkers to model a correlation between a full-length sequence and some certain sequences in the form of a matrix (see FIGS. 1 to 4).

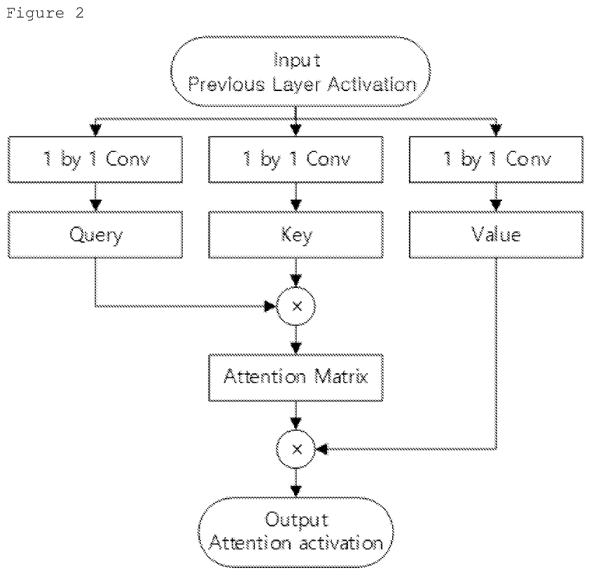

First, a neural network including an attention layer is used to perform learning. The neural network according to one embodiment of the present invention may include a convolutional neural network (CNN). An objective function of the neural network is a disease classification ratio, and cross entropy may be used as a loss function. An attention matrix value that is one of the output values of the attention layer is obtained through a learning process. The matrix is a value representing the size of correlation between a biomarker present at each location and a biomarker present at another location. When the whole neural network may be classified for disease probability with sufficient accuracy, it is possible to deduce a combination of useful biomarkers using the attention matrix. A flowchart for computation of the attention matrix and output values in the attention layer is shown in FIG. 2.

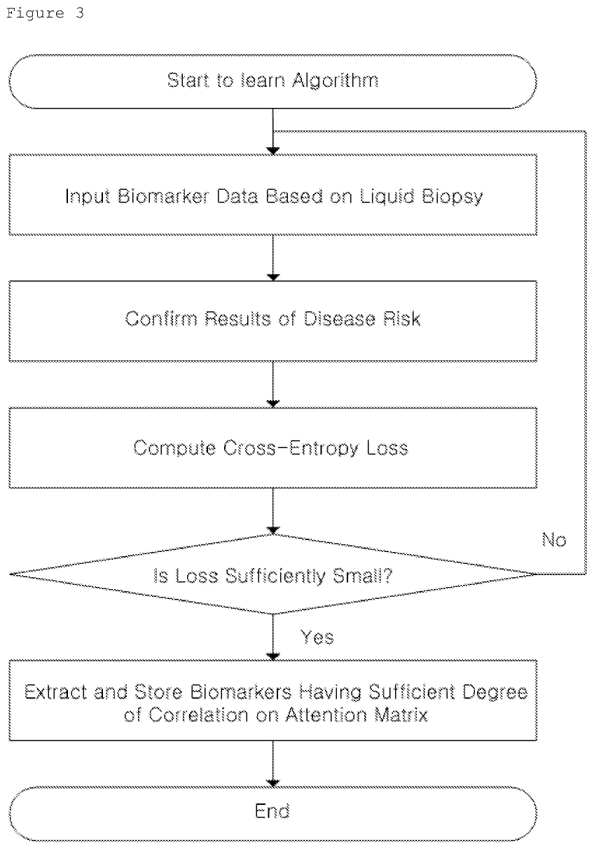

Next, extraction of the biomarkers may be performed in the following steps. When a biomarker is a useless biomarker, first, the biomarker has a low correlation with other biomarkers on the attention matrix. This is because a biomarker is learned not to correlate with other biomarkers as the biomarker is not highly associated with the presence of a disease when the biomarker is a useless biomarker. This is because the biomarker is learned not to correlate with other biomarkers. Therefore, only the biomarkers deduced to have a high correlation on the attention matrix may be selected, and used as the useful biomarkers. Such a method may target the complex biomarkers rather than the single information to remarkably improve the accuracy of prediction of the disease risk. An example of a method for performing a step of extracting biomarkers according to one embodiment of the present invention is shown in FIG. 3.

Furthermore, an attention method will be described. A basic method of attention is to reflect computational outputs by referring to the whole data one more at an encoder at every time step in which the computational outputs are predicted at a decoder. In this case, all of the data are not referred to at the same ratio, but more attentions are paid to a portion of data at the encoder associated with the data to be predicted at the corresponding time step.

A data type composed of key values used in various fields of computer engineering consists of a pair of key and value, and is characterized in that a mapped value may be found through the key.

When attention is expressed as a function, the attention may be expressed as follows.

Attention(Q,K,V)=Attention Value [Equation I]

Q=Query: A hidden state in a decoder cell at a time step (t)

K=Keys: Hidden states in an encoder cell at all the time steps

V=Values: Hidden states in an encoder cell at all the time steps

An attention function is obtained by calculating similarity to all keys for a given query, and the similarity is reflected in each of the values mapped with the keys. Then, all the values in which the similarity is reflected are added up and returned. The value is referred to as an attention value.

A process for calculating an attention distribution using the attention function may be performed using any method, but may, for example, be obtained by softmax function, and the like. In this way, a probability distribution in which the sum of all the values added equals 1 is obtained. In this case, each of the values is referred to as an attention weight.

Based on the results, the attention weight and the hidden state are subjected to a weighted sum at each of encoders to calculate an attention value.

When the attention is applied in the present invention, a method that may calculate an attention score may use a dot function, a scaled dot function, a general function, a concat function, a location-base function, and the like, but the present invention is not limited thereto. It is reasonable that this may be applied without limitation as long as it is a method capable of improving disease predictability according to the present invention.

In the present invention, a process for verifying performance of the disease-specific biomarkers deduced by the method may be preferably further performed. For this purpose, it is more desirable to verify the accuracy of the deduced disease-specific biomarkers by applying the deduced biomarkers to a normal sample or a sample which is not used to detect the biomarkers, and then calculating the classification accuracy.

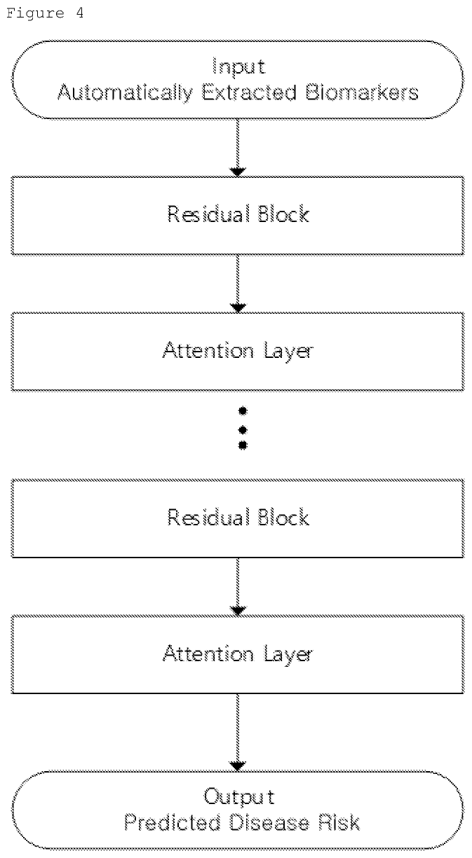

Then, a step of constructing a network model for predicting a disease risk from the disease-specific biomarkers and predicting a disease risk will be described in detail. A schematic diagram of an algorithm for predicting a disease risk according to one embodiment of the present invention is shown in FIG. 4.

In the step of constructing a network model for predicting a disease risk from the disease-specific biomarkers, state changes (such as the onset, progression, and recurrence) of a disease may be constructed in the form of network from the disease-specific biomarkers deduced using the complex genetic information library obtained through analysis of the relation between the types of genetic information, and using an optimization method or a learning method.

A method for constructing the network is not limited as long as it does not hinder achievement of the objects of the present invention, but may include a method for analyzing a change in information in the disease-specific biomarkers deduced according to a change in a certain disease using the genetic information library established by the method. As one example of the analysis, a discontinuous change in expression of the genetic information may be traced so that it can be modeled in the form of a mathematical function. The form of the mathematical function is not particularly limited, but a regression function capable of approximately satisfying the data for the discontinuous change in expression may, for example, be desirably selected as the form of the mathematical function.

Regression analysis methods used to build the regression function are mainly divided into simple regression analysis and multiple regression analysis. In this case, the simple regression analysis may be used to analyze the relation between one dependent variable and one independent variable, and the multiple regression analysis may be used to investigate the relation between one dependent variable and multiple independent variables. A change in expression may be composed of one dependent variable and one independent variable, respectively, so that it can be calculated as a regression function through the simple regression analysis, and one example of the regression function may be an exponential function, and the expression of mi-RNA5 may be modeled as a regression function in the form of a step function.

After the features of the disease-specific biomarkers is mathematically modeled by the method, a genetic information relation network model that is a network model for predicting a disease risk composed of the genetic information may be established to trace a change process of the genetic information according to the main state changes of the disease.

The form of the genetic information relation network model is not limited as long as it does not hinder achievement of the objects of the present invention, but may, for example, be in the form of a statistic disease network in which only a correlation between the types of complex genetic information is applied, or a dynamic disease network in which individual-specific genetic information such as the elapse of time, habits, and the like is added as the variable. Desirably, the form of the genetic information relation network model may be in the form of a dynamic disease network. The form of the network model may be desirably used to trace the constantly changing genetic information features and diagnose and predict a disease.

An algorithm learning method according to one embodiment of the present invention has an advantage in that the disease risk prediction may be smoothly performed under relatively low computational power in a state in which the dimensionality of data is already reduced through the previous step, that is, a step of automatically extracting biomarkers based on machine learning. The objective function of the algorithm for predicting a disease risk is a disease classification ratio as described above, and the cross entropy may be used as the loss. In this case, the softmax function may be used for output activation.

The algorithm for predicting a disease risk computes whether an input value given to an N-dimensional space in the algorithm is closer to a disease sample or a normal sample, and then outputs the input value as a probability value through the output activation using the softmax. In this way, it is possible to predict the disease risk.

In the present invention, the accuracy of the biomarkers and the genetic information relation network model, which is a network model for predicting a disease risk, is not limited, but may be evaluated using the following indicators.
Sensitivity: Sensitivity is a measurement indicator for evaluating whether patients who actually suffer from a disease are well classified, and thus may be defined as TP/(TP+FN) in order to prevent diagnostic failure based on the misdiagnosis. Here, TP represents the number of cases in which patients suffering from the disease are classified into a disease group, and FN represents the number of cases in which the patients suffering from the disease are classified into a normal group. For the biomarkers and the network model for predicting a disease risk, when the sensitivity is desirably greater than or equal to 95%, more desirably greater than or equal to 99%, and most desirably greater than or equal to 99.9%, examination costs may be saved and the feasibility of commercialization may be enhanced. This method is desirable because it increases the cases in which a number of diseases are identified through a single examination using the main genetic information associated with the diseases.
Specificity: Specificity is a measurement indicator for evaluating whether normal persons are actually well classified, and thus is defined as TN/(TN+FP) to prevent unnecessary follow-up examinations according to the pseudo-disease diagnosis. Here, TN represents the number of cases in which normal persons are classified into a normal group, and FP represents the number of cases in which the normal persons are classified into a group of patients suffering from the disease. For the disease risk prediction, the sensitivity or specificity may be used alone or in combination thereof. Among them, the sensitivity may be desirably used together with the specificity because it has higher importance than the specificity in predicting the disease risk.

In the method for predicting a disease risk through analysis of the relation between the types of genetic information according to one embodiment of the present invention, the accuracy of the predicted disease risk is not particularly limited as long as it is in a range of determining a risk of a disease for a small number (i.e., 20 to 35) of the extracted biomarkers. However, when each of the sensitivity and the specificity is greater than or equal to 75%, when each of the sensitivity and the specificity is desirably greater than or equal to 80%, and when each of the sensitivity and the specificity is more desirably greater than or equal to 90%, examination costs may be saved and the feasibility of commercialization may be enhanced. This method is desirable because it increases the cases in which a number of diseases are identified through a single examination using the main genetic information associated with the diseases.

In the present invention, any diseases may be applied as the disease as long as they may be applied to deduce the biomarkers. In this case, the disease may be caused by cognitive function- and/or memory-related impairments. As one specific example, the disease may include one or more selected from the group consisting of Alzheimer's disease, Huntington's disease, Parkinson's disease, amyotrophic lateral sclerosis, or the like, but the present invention is not limited thereto.

Also, the present invention provides a disease-specific biomarker deduce by the method for predicting a disease risk through analysis of the relation between the types of genetic information.

In the present invention, it is expected that the deduced biomarker may be effectively used to manufacture medical equipment including diagnostic chips and a terminal and judge the prognosis of the disease through commercialization toward the disease diagnosis service.

Hereinafter, the contents of the present invention will be described in further detail with reference to examples thereof. It should be understood that the following examples are illustrative only to describe the present invention in more detail, but are not intended to limit the scope of the present invention

### EXPERIMENTAL MATERIALS

RNA sequence specimen data associated with mild cognitive impairments and Alzheimer's disease were acquired from NCBI AddNeuroMed database (NCBI GEO Datasets), and used.

GEO dataset serial number: GSE63063, GSE63060

### [Example 1] Extraction of Biomarkers for Identifying Patient with Mild Cognitive Impairment

The aforementioned attention layer and attention model were applied to the GSE63063 and GSE63060 data to extract RNA sequence biomarkers based on a liquid biopsy. The listing is shown in the following Table 1.

TABLE 1
Biomarkers for identifying patients
with mild cognitive impairment

| | Biomarkers |
|---|---|
| Example 1 | 6973 ILMN_1343291 |
| | 9069 ILMN_1343295 |
| | 33349 ILMN_1651209 |
| | 22372 ILMN_1651221 |
| | 32091 ILMN_1651228 |
| | 19428 ILMN_1651229 |
| | 521 ILMN_1651235 |
| | 4554 ILMN_1651237 |
| | 25590 ILMN_1651254 |
| | 8468 ILMN_1651259 |
| | 10420 ILMN_1651262 |
| | 25548 ILMN_1651268 |
| | 33712 ILMN_1651278 |
| | 23081 ILMN_1651279 |
| | 5202 ILMN_1651282 |
| | 1997 ILMN_1651285 |
| | 21250 ILMN_1651288 |
| | 20935 ILMN_1651296 |
| | 10368 ILMN_1651315 |
| | 4348 ILMN_1651316 |
| | 20575 ILMN_1651328 |
| | 19722 ILMN_1651330 |
| | 26735 ILMN_1651336 |
| | 21646 ILMN_1651341 |
| | 19512 ILMN_1651343 |
| | 35173 ILMN_1651346 |
| | 32759 ILMN_1651347 |
| | 34075 ILMN_1651354 |
| | 10037 ILMN_1651358 |
| | 29089 ILMN_1651364 |

In comparison with the results, the conventional variational dropout mode had a drawback in that the biomarkers diverged during the process for extracting the biomarkers when the conventional variational dropout mode was applied to extract the biomarkers. However, it was confirmed that, when the attention model according to the present invention was used, the attention model had a converging effect during the biomarker extraction step.

### [Example 2] Prediction of Accuracy of Deduced Biomarkers

A computation for predicting a disease risk using the biomarkers deduced in the same manner as in Example 1 was performed. The results are as follows.

The computation was performed using a 100-fold cross validation method so that the measurement results of each of the sensitivity and specificity were not the results specific to certain learning sets but were the common results for the algorithm itself.

Fully connected layers were composed of 1,024, 512, 256, and 128 nodes, and the last layer was composed to classify the disease probabilities using a readout layer and softmax activation.

### [Comparative Example 1] Using Only CNN without Extracting Biomarkers

A disease risk was predicted in the other same conditions except that the CNN was applied to the whole genomic data to predict the accuracy without applying the conventional variational dropout method to the GSE63063 and GSE63060 datasets.

### [Comparative Example 2] Extracting Biomarkers and Using CNN

The conventional variational dropout method was applied to the GSE63063 and GSE63060 datasets to extract useful biomarkers from the genomic data, and the CNN based on the extracted biomarkers was used to predict a disease risk.

The results are shown in the following Table 2.

TABLE 2
Results of disease risk prediction
according to experimental groups

| Experimental | | | Number of |
|---|---|---|---|
| groups | Sensitivity | Specificity | biomarkers |
| Examples 1 | 83.0% | 79.0% | 30 |
| and 2 | | | |
| Comparative | 80.0% | 77.0% | tens of thousands |
| Example 1 | | | |
| Comparative | 50.0% | 50.0% | Failed to extract |
| Example 2 | | | biomarkers |
| | | | (divergence) |

From the results, it was confirmed that the method for predicting a disease risk through analysis of the relation between the types of genetic information according to the present invention provided the biomarkers having high accuracy to mild cognitive impairments which were difficult to distinguish from the existing diseases, and also had a sensitivity of 83.0% and a specificity of 79.0% using only the 30 biomarkers.

On the other hand, when the variational dropout method was used instead of the method for extracting biomarkers according to the present invention, several tens of thousands of biomarkers had to be applied to the variational dropout method. Therefore, the variational dropout method had a problem in that it took a long time to deduce the results due to a highly increased computation time. Nevertheless, it was confirmed that the variational dropout method had a problem such as low sensitivity and specificity, compared to that of the present invention. When the method for extracting biomarkers was not used, the extraction of the biomarkers did not converge but did diverge, resulting in failure to extract the biomarkers. As a result, it was confirmed that it had a problem in that the sensitivity and specificity were fixed to 50%.

That is, based on the results, the methods for extracting biomarkers and predicting a disease risk according to the present invention were applied to predict mild cognitive impairments. As a result, it was confirmed that the methods were able to save examination costs and enhance the feasibility of commercialization, and enabled identification of diseases in a rapid period of time through a single examination using the main genetic information. From the results, it was revealed that the methods for extracting biomarkers and predicting a disease risk according to the present invention were used as the diagnostic technology that satisfied a commercially available level of accuracy and economic feasibility. Therefore, the present invention has been completed based on these facts.

It was confirmed that the method for predicting or diagnosing a disease risk using the biomarkers extracted from the liquid biological specimen developed according to the present invention applies an improved network analysis and learning method as compared to conventional methods, and thus enables disease-related diagnosis which shows high sensitivity and specificity even when only a few biomarkers are applied, which indicates that the method of the present invention shows superior extraction performance, compared to the methods using conventional variational dropout-based biomarker extraction.

What is claimed is:

1. A method for predicting a disease risk through analysis of the relation between the types of genetic information, the method comprising:
extracting types of complex genetic information from specimens of a patient and a normal person;
constructing a complex genetic information library through information comparison/analysis between the types of complex genetic information;
extracting disease-specific biomarkers from the complex genetic information library; and
constructing a network model for predicting a disease risk from the disease-specific biomarkers and predicting a disease risk.

2. The method of claim 1, wherein the types of complex genetic information comprise information on expression of genetic information of any one or two or more selected from the group consisting of DNAs, RNAs, and proteins, or information on syntheses thereof.

3. The method of claim 1, wherein the complex genetic information library is constructed by deducing the types of complex genetic information using a statistical analysis or optimization method.

4. The method of claim 1, further comprising, when the disease-specific biomarkers are extracted:
securing information on a base sequence of the corresponding genetic information to extract genetic information variations including single nucleotide polymorphisms (including addition, deletion, or substitution of the base sequence), or copy number variations.

5. The method of claim 1, wherein the relation between a disease and the types of complex genetic information present in the complex genetic information library is analyzed using an optimization method or learning method to extract a biomarker associated with the disease.

6. The method of claim 1, wherein a statistic disease network model or a dynamic disease network model is constructed based on the disease-specific biomarkers.

7. The method of claim 5, wherein the optimization method is selected from the group consisting of a simulated annealing method, a genetic algorithm, a tap search method, a simulated evolution, a probabilistic evolution method.

8. The method of claim 5, wherein the learning method is selected from the group consisting of a neural network and deep learning.

9. The method of claim 8, wherein the neural network is selected from the group consisting of a convolutional neural network (CNN) and a recurrent neural network (RNN).

10. The method of claim 5, wherein an attention model using an attention layer is applied upon extraction of the biomarkers to model a correlation between a full-length sequence and some certain sequences in the form of a matrix.

11. The method of claim 1, wherein the accuracy of the predicted disease risk is such that sensitivity and specificity to the 20 to 35 extracted biomarkers is greater than or equal to 80%, respectively.

12. The method of claim 1, wherein the disease is caused by cognitive function- and/or memory-related impairments.

13. The method of claim 12, wherein the disease is Alzheimer's disease, Huntington's disease, Parkinson's disease, or amyotrophic lateral sclerosis.

14. A disease-specific biomarker extracted by the method for predicting a disease risk as defined in claim 1.
